(19) United States
(12) Patent Application Publication
(10) Pub. No.: US 2026/0237464 A1
(43) Pub. Date: Aug. 13, 2026

(54) COMPUTER-IMPLEMENTED METHOD FOR ANALYSING WATER QUALITY

(71) Applicant: SUEZ INTERNATIONAL, Puteaux (FR)

(72) Inventors: Agnès PETRAU, BRISCOUS (FR); Maialen SAGARDUY, Cambo-les-Bains (FR)

(21) Appl. No.: 19/150,977

(22) PCT Filed: Jan. 26, 2024
(86) PCT No.: PCT/EP2024/051980

§ 371 Date: Jul. 25, 2025

(30) Foreign Application Priority Data
Jan. 27, 2023 (FR) FR2300799

(51) Int. Cl. G16B 40/10; C12Q 1/06; C12Q 1/6851; C12Q 1/686; C12Q 1/689; G01N 33/18; G06N 20/00; G16B 25/20; G16B 40/20

(52) U.S. Cl. CPC G16B 40/10; C12Q 1/06; C12Q 1/6851; C12Q 1/686; C12Q 1/689; G01N 33/1826; G06N 20/00; G16B 25/20; G16B 40/20

(57) ABSTRACT

A computer-implemented method (1) for estimating a parameter representative of a quantity of the viable culturable bacteria indicative of fecal contamination in a collected water sample, comprising:
a step (10) of acquiring values representative of a plurality of environmental parameters associated with collecting the water sample;
a step (11) of quantifying the viable bacteria present in the collected water sample;
a step (12) of implementing a statistical regression module (50) suitable for estimating a value representative of the quantity of the viable culturable bacteria according to the values representative of the acquired environmental parameters and the quantification of the viable bacteria in the water sample.

The invention relates to a computer-implemented method for analyzing water quality. More particularly, the invention relates to a computer-implemented method for estimating a parameter representative of a quantity of the viable culturable bacteria indicative of fecal contamination in a collected water sample.

Monitoring bacteria indicative of fecal contamination in recreational waters is an important public health measure to minimize waterborne diseases. Thus, the European Bathing Water Directive 2006/7/EC imposes the monitoring of Escherichia coli and intestinal enterococci bacteria, whose presence in water indicates fecal contamination. Although these microbial germs do not themselves pose a danger to bathers at the levels usually detected, their presence can indicate the simultaneous presence of pathogenic germs. It is therefore relatively important to measure the concentration of these bacteria in water.

Several measurement methods are known to quantify the presence of these bacteria in water.

Notably, a miniaturized method using microplates for inoculation in liquid medium (ISO 9308-3 and 7899-1 for Escherichia coli and enterococci respectively) is known. This standardized method is based on the ability of these bacteria to multiply in a suitable medium. However, it has the disadvantage of a long response time, generally between 36 h to 72 h, which is relatively unsuitable for active management of bathing waters. Indeed, water quality degradations are generally temporary and dissipate quickly depending on hydro-climatic conditions. Therefore, water quality analysis measurements must be obtained as quickly as possible to avoid exposing the public to pathogens, especially in pools and at beaches.

In recent decades, new methods have been developed to reduce this response time and allow decisions to be made within a few hours after sample collection.

The enzymatic methods developed in recent years are based on the same principle as the previous miniaturized microplate method. Each of the two bacteria has a quasi-specific enzyme: β-D-glucuronidase for E. coli and β-D-glucosidase for enterococci. These enzymes can hydrolyze artificial compounds, releasing a colored or fluorescent product that allows visual quantification of the bacteria initially present in the sample. Many detection protocols and devices have been developed in recent years based on this principle. The response times of these different devices are shorter than those observed for the miniaturized microplate technique but remain too long to allow active management of bathing waters. These times are indeed between 12 h to 24 h depending on the method used to obtain a result <100 bacteria/100 ml.

Different methods have been developed to further reduce analysis time. In particular, the so-called polymerase chain reaction techniques, generally abbreviated as PCR, from the English Polymerase Chain Reaction, are known, which allow rapid and relevant results to be obtained.

The quantitative PCR method, known as qPCR, is based on the detection and quantification of a gene section of the target bacteria. Bacteria quantification is performed using fluorescent molecules or probes that are released in the same proportion as the targeted genes after amplification cycles. It is a sensitive, specific method, also detecting bacteria with repressed or unexpressed glucuronidase or glucosidase activity and requiring less than 3 hours to provide a result.

However, the problem with this type of method is that it is not discriminating with respect to the different physiological states of bacteria that can be encountered in a bacterial population: culturable, viable, senescent, and dead bacteria.

The RT-qPCR method (Reverse Transcriptase-quantitative Polymerase Chain Reaction) relies on the extraction and amplification of ribonucleic acid (or RNA), with RNA being more representative of cellular viability than DNA. The method consists of extracting the target RNA, then transcribing it into a complementary DNA strand. This complementary DNA strand, frequently abbreviated as cDNA, then serves as a template for a classic quantitative PCR reaction. It is a sensitive and specific method that only detects viable bacteria and allows a result on the two regulatory bacteria E. coli and fecal enterococci to be obtained in less than 3 hours and up to around 40 samples to be taken simultaneously.

However, the RT-qPCR method has the disadvantage of not discriminating between viable culturable and viable non-culturable bacterial populations, whereas it is useful to know only the viable culturable population in certain regulatory contexts.

A viable non-culturable bacterium is a living bacterium that has lost its ability to multiply on culture media. Bacterial cells enter a viable non-culturable state when subjected to stress related to environmental factors in their environment.

In many regulatory frameworks, these viable non-culturable bacteria are excluded from the concentrations considered for deciding whether a “recreational” body of water, such as a pool or the sea near a beach, should be closed to the public due to bacterial pollution.

There is therefore a need for a rapid, simple-to-use quantification method for bacteria, particularly Escherichia coli and intestinal enterococci, allowing quantification of viable culturable bacterial populations.

To this end, a computer-implemented method is proposed for estimating a parameter representative of a quantity of the viable culturable bacteria indicative of fecal contamination in a collected water sample, comprising:
A step of acquiring values representative of a plurality of environmental parameters associated with collecting the water sample;
A step of quantifying the viable bacteria present in the collected water sample;
A step of implementing a statistical regression module suitable for estimating a value representative of the quantity of the viable culturable bacteria according to the values representative of the acquired environmental parameters and the quantification of the viable bacteria in the water sample.

Thus, a rapid and robust estimate of a quantity of the viable culturable bacteria in a collected water sample can be made.

In particular, the method then implements a display step or a transmission step of the estimated value representative of the quantity of the viable culturable bacteria.

In particular, the display can be performed, for example, on a human-machine interface, such as a screen, on any type of digital medium or on a printed medium.

The transmission of the estimated value representative of the quantity of the viable culturable bacteria can be a digital transmission, for example, via a wired or wireless communication network, to a remote server or a database.

Advantageously and without limitation, the step of quantifying the viable bacteria present in the collected water sample comprises the implementation of a polymerase chain reaction method, in particular a real-time quantitative polymerase chain reaction. Thus, a particularly rapid quantification result can be obtained.

In particular, the bacteria indicative of fecal contamination comprise a fecal enterococcus bacterium and/or an Escherichia coli bacterium.

Advantageously, the environmental parameters are chosen from: a parameter representative of the water's salinity, a parameter representative of the water's turbidity, a parameter representative of the water height, a parameter representative of the wind, a parameter representative of solar radiation, a parameter representative of rainfall, and a parameter representative of swell. These parameters are particularly relevant for allowing a reliable estimate by means of the regression module.

In particular, for each environmental parameter, an instantaneous value, or an average value over a measurement period of the environmental parameter, or a cumulative value of the measurements made over a measurement period of the environmental parameter, or a time series representative of a measurement period of the environmental parameter is acquired.

Advantageously, the statistical regression module is a machine learning module trained from a training data set, wherein each training data associates:
Values representative of environmental parameters associated with a collected water sample;
A value representative of the quantity of the viable bacteria, comprising viable culturable and non-culturable bacteria in the sample; and
A value representative of a quantity of the viable culturable bacteria in the collected water sample;

The machine learning module being trained to infer, for each data set, the value representative of a quantity of the viable culturable bacteria from the associated environmental parameters and the value representative of the quantity of the viable bacteria, comprising viable culturable and non-culturable bacteria.

In particular, the machine learning module is chosen from a gradient boosting machine learning module, a support vector machine, an artificial neural network, a k-nearest neighbors method, a decision tree module, or a random forest machine learning module.

The invention also relates to a computer-implemented training method for a machine learning module for implementing a method as described above, comprising:

A step of providing a training data set, wherein each training data associates:
Values representative of environmental parameters associated with collecting a water sample;
A value representative of a quantity of the viable bacteria, comprising viable culturable and non-culturable bacteria; and
A value representative of a quantity of the viable non-culturable bacteria in the water sample;
A step of training the machine learning module to infer, for each data set, the value representative of a quantity of the viable culturable bacteria from the associated environmental parameters and the value representative of the quantity of the viable bacteria, comprising viable culturable and non-culturable bacteria.

The invention also relates to a device for estimating a parameter representative of a quantity of the viable culturable bacteria indicative of fecal contamination in a collected water sample, comprising:
Means for acquiring a plurality of values representative of environmental parameters associated with collecting the water sample;
Means for acquiring a quantification of the viable bacteria present in the collected water sample;
An implementation unit of a statistical regression module suitable for estimating a value representative of the quantity of the viable culturable bacteria according to the values representative of the acquired environmental parameters and the quantification of viable bacteria in the water sample.

The invention also relates to a training device for a machine learning module for implementing an estimation method as described above, comprising:
Means for providing a training data set, wherein each training data associates:
   Values representative of environmental parameters associated with a water sample;
   A value representative of a quantity of the viable bacteria, comprising viable culturable and non-culturable bacteria; and
   A value representative of a quantity of the viable non-culturable bacteria in the water sample;
A training unit for the machine learning module to infer, for each data set, the value representative of a quantity of the viable culturable bacteria from the associated environmental parameters and the value representative of a quantity of the viable bacteria, comprising viable culturable and non-culturable bacteria.

Other features and advantages of the invention will emerge from the reading of the description made below of a particular embodiment of the invention, given as an indication, but not limiting, with reference to the appended drawings on which:

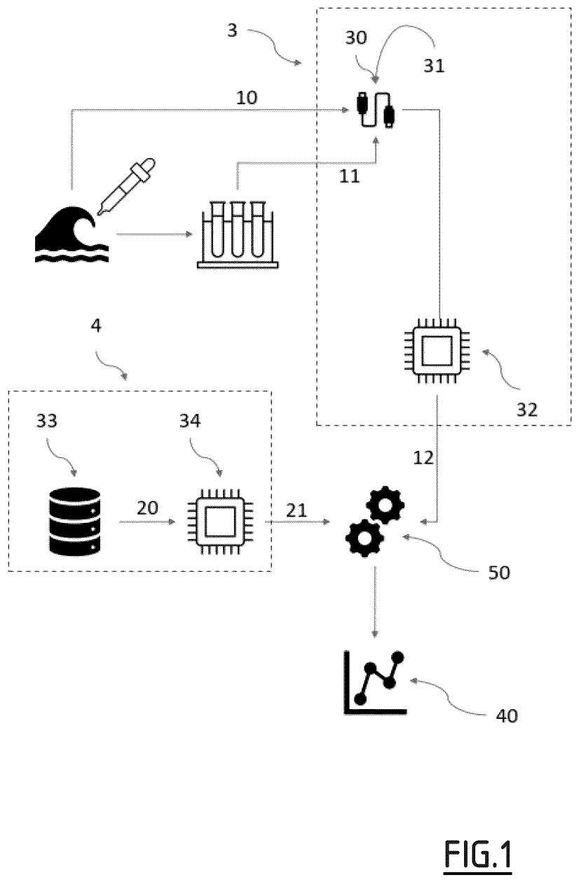

FIG. 1 is a schematic representation of the devices for implementing the estimation method according to the invention and the training method according to the invention;

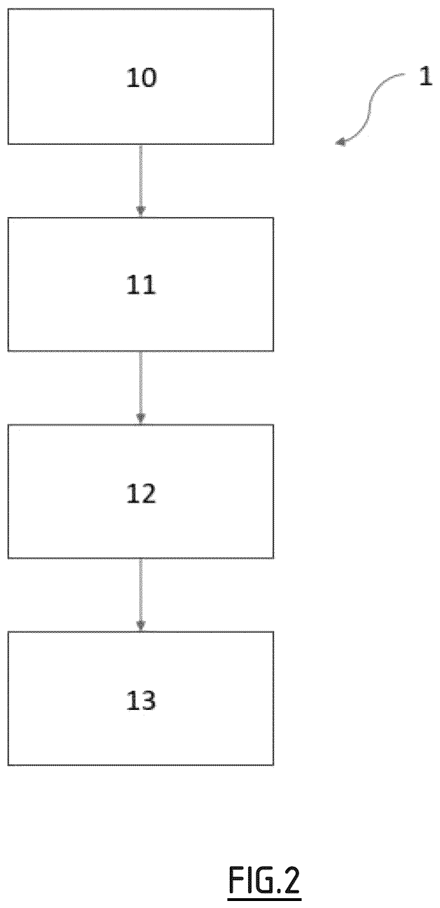

FIG. 2 is a flowchart of the steps for the estimation method according to the main embodiment of the invention; and

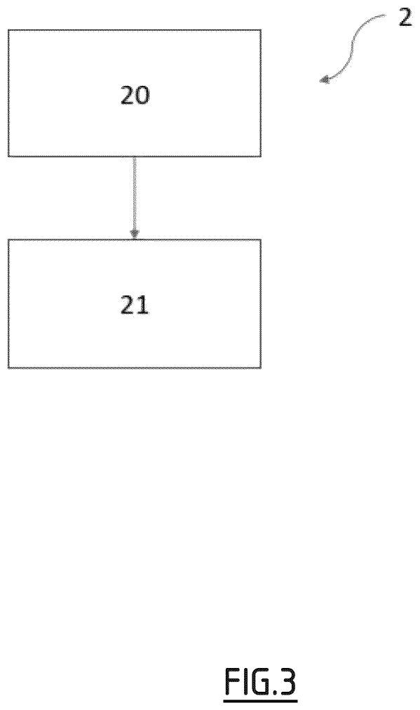

FIG. 3 is a flowchart of the steps for the training method according to the main embodiment of the invention.

FIGS. 1 to 3 relating to the same embodiment will be commented on simultaneously.

The invention relates to a computer-implemented method 1 for estimating the quantity of the viable culturable bacteria in a collected water sample.

In the main embodiment of the invention, method 1 aims to detect E. coli or intestinal enterococci bacteria, but it could be adapted to detect any other bacteria indicative of fecal contamination.

Thus, in the sense of the invention, the general term viable bacteria means viable bacteria indicative of fecal contamination.

Generally, method 1 according to the invention is adapted in its implementation for a type of bacteria and a type of sampled water.

Thus, as an example, and without limitation, several methods 1 can be implemented: a method 1 according to the invention to quantify viable culturable E. coli bacteria and a method 1 according to the invention to quantify viable culturable intestinal enterococci bacteria. The two obtained quantifications enable verification of whether the regulatory health conditions have been met.

Similarly, different methods can be implemented depending on the water sampling environment. For example, separate methods can be provided for both seawater and freshwater environments.

Thus, in an example embodiment of the invention, four different methods 1 can be provided: a method 1 for quantifying E. Coli for seawater, a method 1 for quantifying E. Coli for freshwater, a method 1 for quantifying intestinal enterococci for seawater, and a method 1 for quantifying intestinal enterococci for freshwater.

However, method 1 in its main embodiment, regardless of the type of bacteria and the type of sampled water, is implemented as follows.

Firstly, a step of acquiring 10 values representative of environmental parameters associated with collecting the water sample is implemented.

The set of selected environmental parameters aims to represent the environmental conditions in which the water was collected.

The values representative of these environmental parameters are, in the main embodiment of the invention, average values of measurements taken over defined measurement periods or cumulative values over a defined measurement period, depending on the environmental parameters, or instantaneous values.

Instantaneous values are preferably acquired at the time of collection, for example for salinity or turbidity.

Depending on the parameters, an average value can also be acquired, for example for swell, wind, or cumulative values, for example for rain or solar radiation.

However, in an alternative embodiment, the values could be time series, or a combination of time series, instantaneous values, average values, and cumulative values.

For the rest of the description, the “measurement period” is defined as a time period starting before the water collection and ending at the time of collection. Thus, a 12-hour measurement period is understood as the acquisition of measurement data starting 12 hours before the collection and ending at the time of collection.

However, the invention is not limited to this sole interpretation of the measurement period, as measurement periods can notably be offset to end before or after the collection.

Indeed, time-offset measurements, for example ending one hour before or one hour after the collection, as an example, could prove just as reliable within the framework of the invention.

Each measured environmental parameter can be measured over its own measurement period, possibly different from other measured environmental parameters.

The invention is not limited to a fixed list of environmental parameters, but in the main embodiment of the invention, the following environmental parameters are measured:
the salinity of the water, measured at the time of collection. Generally, salinity is measured using a conductivity meter or a salinity probe. These devices measure the water's ability to conduct electricity, which is directly related to the concentration of dissolved salts in the water. The higher the salt concentration, the more conductive the water is. Salinity is generally expressed in electrical conductivity units (EC), in conductivity units in microsiemens per centimeter (μS/cm). It should be noted that water temperature affects conductivity, and it is generally necessary to correct conductivity measurements based on temperature to obtain an accurate salinity measurement.
the turbidity of the water, measured at the time of collection. Turbidity is generally measured using a turbidimeter, which implements a light beam through the water sample and measures the amount of light that is reflected or absorbed by suspended particles in the water. The more particles there are, the more light is reflected and absorbed, and the higher the turbidity. This turbidity is generally quantified by quantities dimensioned in NTU units (from the English Nephelometric Turbidity Units) or FTU (Formazin Turbidity Units) or in the amount of suspended matter generally quantified in mg/l or ppm.
the wind, measured or modeled, and averaged over a measurement period that can range from 1 h to 24 h, in this embodiment the measurement period being 12 h and 24 h before the collection. To quantify the wind associated with the sample, a wind speed, in meters/second, is acquired. However, alternatively, a wind speed and a general wind direction value, in degrees, could be combined. Generally, these data representative of wind can be measured directly near the water sample collection site or acquired via external data from remote databases, or extracted from meteorological modeling and prediction systems.
rainfall, particularly values representing cumulative precipitation and maximum hourly intensities over measurement periods ranging from 1 to 72 hours before each collection.
solar radiation, calculated as the sum of solar radiation (in Watts/m2), over a measurement period generally starting at midnight the day before the collection day and ending at the time the water sample is collected.
waves, or swell, when the collection is made in an area with significant waves, such as an oceanic area. In this embodiment of the invention, waves, or swell, are measured in the case of seawater measurement. However, the invention is not limited to this particular choice. The average wave height is measured or modeled at the closest point to the collection point, over a measurement period that can range from 1 to 24 h before the collection, here 6 h, 12 h, and 24 h. Generally, we calculate the average wave height by modeling it at the collection point, based on data obtained from external sources. In an alternative embodiment, the data representative of waves could combine both an average height and a wave direction, obtained for example from digital modeling.
the water height, related to tides, acquired at the time of collection, is derived here from a water height prediction model, for example, a model specifically made for the measurement site, generally measured in meters.

Additionally, according to a particular embodiment, location values for the collection, such as geolocation coordinates, or a name, such as a city name or a beach name, can be added to the environmental parameters.

In an alternative embodiment, the water temperature can also be chosen among the environmental parameters.

A step of quantifying 11 the viable bacteria indicative of fecal contamination present in the collected water sample is also implemented.

In this embodiment, the quantified bacteria are Escherichia coli, also called E. coli, and intestinal enterococci. However, the invention is not limited to these two bacteria, which are nevertheless two particularly advantageous indicators of bacterial water pollution.

This quantification step 11 is not necessarily performed after the acquisition step 10. It can be implemented before or in parallel with the acquisition step 10.

This quantification step 11 is in our embodiment of the invention implemented according to a method described in document EP3599283A1 “Method for assessing fecal pollution in water”, which discloses an RT-qPCR method for quantifying the sought bacteria indicative of fecal contamination.

The invention is not limited to this particular method, and any rapid quantification method, allowing the detection of viable bacteria indicative of fecal contamination, such as E. coli or fecal enterococci, can be implemented.

The quantification step 11 allows the cycle threshold value to be obtained, abbreviated as Ct for “cycle threshold” in English, which corresponds to the number of amplification cycles required to reach a fluorescence threshold value. This intersection point confirms the presence of the target in the sample and determines its concentration. The Ct is inversely proportional to the amount of genetic material present in the sample.

This cycle threshold value can then be transformed, by regression, into a bacterial count.

However, regardless of the technique used, it is well known that in rapid or semi-rapid approaches, for example, over durations of less than 12 h, the obtained quantifications do not allow discrimination between viable culturable and viable non-culturable bacteria.

However, for regulatory needs, it is necessary to know only the viable culturable bacteria that are considered in recreational bathing water regulations.

To this end, a statistical regression module 12 is then implemented to estimate a value representative of the quantity of the viable culturable bacteria based on the values representative of the acquired environmental parameters and the quantity of the viable bacteria in the water sample.

In the main embodiment of the invention, the regression module is suitable for estimating a count of viable culturable bacteria, here dimensioned in the most probable number per 100 ml, abbreviated as MPN per 100 ml. However, any other unit could be used.

In an alternative embodiment, a regression module suitable for providing another quantification or count value could be used.

In this embodiment of the invention, this statistical regression module 50 is a gradient boosting machine learning module 50.

Gradient boosting is a machine learning algorithm mainly used to solve regression problems. It uses an optimization technique consisting of iteratively adding new models to an existing set of models.

Each new model is generated to correct the errors made by the previous models. The models are generally decision trees but can be other types of models.

Gradient boosting is particularly effective for regression tasks because it allows very accurate models to be generated by combining many weaker base models.

However, the invention is not limited to this particular machine learning module 50. Other regression techniques can be employed, such as a support vector machine, known as SVM, an artificial neural network, a k-nearest neighbors method, a decision tree module, or a random forest machine learning module.

Advantageously, a machine learning module 50 specifically trained for the type of bacteria considered is generally implemented, allowing more accurate results to be obtained for each type of bacteria.

In particular, with European regulations requiring these two types of bacteria to be monitored, in a particular implementation of the invention, we implement two distinct detection methods 1, each comprising a separately trained machine learning module.

Thus, a machine learning module 50 trained for E. coli bacteria and/or a machine learning module 50 trained for intestinal enterococci bacteria is implemented, depending on the bacteria to be monitored.

Furthermore, it is also possible to train a machine learning module 50 by type of bacteria and by type of sampling environment, such as a marine environment or a freshwater environment.

Indeed, in a non-saline water environment, the training parameters may be different, for example, swell or waves may not be considered.

In the sense of the invention, the term value representative of the quantity of the viable culturable bacteria can be an indicator of the quantity of the viable culturable bacteria, such as a count of the quantity of the viable culturable bacteria.

The quantity of the viable culturable bacteria being directly calculable from the quantity of the viable non-culturable bacteria, by subtracting from the total count that can be calculated from the cycle threshold value Ct obtained during the quantification step 11, the value representative of the quantity of the viable culturable bacteria can also be a count of the quantity of the viable non-culturable bacteria.

Method 1 optionally implements a display and/or transmission step 40 of the estimated value representative of the quantity of the viable culturable bacteria, the display and transmission can be performed alone or in combination.

In particular, display 40 can be performed, for example, on a human-machine interface, such as a screen, on any type of digital medium or on a printed medium.

Transmission 40 of the estimated value representative of the quantity of the viable culturable bacteria can be a digital transmission, for example, via a wired or wireless communication network, to a remote server or a database. For example, via an intranet network, or via the internet.

The invention, according to a particular embodiment of the invention, comprises a comparison step between the determined quantity of the viable culturable bacteria and a predetermined threshold value, such as a pollution threshold value.

In this particular embodiment, an alert step can also be implemented for a user, such as a message on a human-machine interface, sending a written message, such as an email, communicating an alert frame to a remote server, when the value representative of the quantity of the viable culturable bacteria is above a determined threshold, such as an alert threshold, to indicate contamination by a bacterium indicative of fecal contamination.

However, to implement method 1 according to the invention, when is the method relates to a machine learning module 50, it is necessary to train the module before the method can be implemented.

To this end, the invention also comprises a training method 2 for the machine learning module 50.

This training method firstly comprises a step of providing 20 a training data set, wherein each training data associates:
Values representative of environmental parameters associated with collecting a water sample;
A value representative of a quantity of the viable bacteria, comprising viable culturable and non-culturable bacteria; and
A value representative of a quantity of the viable culturable bacteria in the water sample;

The values representative of the environmental parameters associated with collecting the water sample are the same as those for the estimation method 1 described above. It is indeed necessary to train the machine learning module on the same parameters that will then be provided in the steps for implementing the machine learning module.

In the main embodiment of the invention, the training data are provided to train the machine learning module 50 specifically for a type of bacteria indicative of fecal contamination, particularly E. coli, or intestinal enterococci bacteria. In this embodiment, moreover, the module is specifically trained for a type of collected water: seawater or freshwater.

However, the invention is not limited to such training data, and the model could be trained for several types of bacteria simultaneously and/or for several types of water simultaneously.

The method then comprises a training step 21 for the machine learning module 50 to infer, for each data set, the value representative of a quantity of the viable culturable bacteria from the associated environmental parameters and the value representative of the quantity of the viable bacteria, comprising viable culturable and non-culturable bacteria.

This training step is performed, within the framework of gradient boosting, as follows:

Firstly, an initialization step is implemented, during which a base model, here a decision tree, is trained on the training data to produce predictions; here the prediction is the value representative of a quantity of the viable culturable bacteria in the water sample.

A step of calculating errors. The errors made by the base model are calculated by comparing the obtained predictions with the actual values.

Then, a training step for the following models is carried out: a new model is trained to correct the errors made by the previous model using an optimization technique, such as a gradient descent algorithm.

Then, a combination of the models is carried out. The trained models are combined to produce a final prediction. This combination can be done in different ways, such as using a weighted average or using a majority vote.

These steps are then repeated iteratively, for example, over a predetermined number of iterations, or until a defined error rate is obtained.

The features of the previously described embodiments can be taken in isolation or in combination according to all technically possible combinations.

The estimation method according to the invention is implemented by a device 3 that comprises acquisition means 30 of the values representative of the environmental parameters, such as input-output communication means 30 that can be of any known type, Ethernet port, COM port, USB port, wired or wireless communication device, and storage means suitable for storing in memory, either in RAM or on a storage medium, the acquired representative values.

Device 3, here a computer, also comprises acquisition means 31 of a quantification of the viable bacteria present in the collected water sample.

This quantification 31 is generally performed by implementing RT-qPCR quantification means.

The results of this RT-qPCR quantification are then acquired by device 3 by means of acquisition means 31, which can be identical or distinct from acquisition means 30 of the values representative of the environmental parameters.

Device 3 also comprises an implementation unit for a statistical regression module 50 as described above.

Here, the implementation unit 32 is a processor 32, or any digital calculator suitable for implementing a statistical regression module 50 according to the invention.

The training method 2 is implemented by a training device 4, such as a computer, comprising provision means 33 of a training data set, such as a storage medium, for example, a hard drive or a flash memory or any input-output communication means suitable for transmitting training data.

The training device 4, here a computer, comprises a training unit 34 of the machine learning module 50.

The training unit 34 is a processor, a calculator, or any digital set suitable for training a machine learning module 50.

Furthermore, the training unit 34 can implement the training steps for the training method 2 with parallel computing processors, such as GPUs implemented in a general computing context, known in English by the acronym GPGPU, for General-Purpose computing on Graphics Processing Units. This listing of claims will replace all prior versions, and listings, of claims in the application:

1-10. (canceled)

11. A computer-implemented method for estimating a parameter representative of a quantity of viable culturable bacteria indicative of fecal contamination in a collected water sample, comprising:
a step of acquiring values representative of a plurality of environmental parameters associated with collecting the water sample;
a step of quantifying the viable bacteria indicative of fecal contamination present in the collected water sample;
a step of implementing a statistical regression module suitable for estimating a value representative of the quantity of the viable culturable bacteria indicative of fecal contamination according to the values representative of the acquired environmental parameters and the quantification of viable bacteria in the water sample.

12. The method according to claim 11, wherein the step of quantifying the viable bacteria indicative of fecal contamination present in the collected water sample comprises the implementation of a polymerase chain reaction method.

13. The method according to claim 12, wherein the step of quantifying the viable bacteria indicative of fecal contamination present in the collected water sample comprises the implementation of a real-time quantitative polymerase chain reaction.

14. The method according to claim 11, wherein the bacteria indicative of fecal contamination comprise a fecal enterococcus bacterium and/or an Escherichia coli bacterium.

15. The method according to claim 11, wherein the environmental parameters are chosen from: a parameter representative of the water's salinity, a parameter representative of the water's turbidity, a parameter representative of the water height, a parameter representative of the wind, a parameter representative of solar radiation, a parameter representative of rainfall, and a parameter representative of swell.

16. The method according to claim 11, wherein, for each environmental parameter, an instantaneous value, an average value over a measurement period of the environmental parameter, or a cumulative value of the measurements made over a measurement period of the environmental parameter, or a time series representative of a measurement period of the environmental parameter is acquired.

17. The method according to claim 11, wherein the statistical regression module is a machine learning module trained from a training data set, wherein each training data associates:
Values representative of environmental parameters associated with a collected water sample;
A value representative of a quantity of the viable bacteria, comprising viable culturable and non-culturable bacteria in the sample; and
A value representative of a quantity of the viable culturable bacteria in the collected water sample;
The machine learning module being trained to infer, for each data set, the value representative of the quantity of the viable culturable bacteria from the associated environmental parameters and the value representative of the quantity of the viable bacteria, comprising viable culturable and non-culturable bacteria.

18. The method according to claim 17, wherein the machine learning module is chosen from a gradient boosting machine learning module, a support vector machine, an artificial neural network, a k-nearest neighbors method, a decision tree module, or a random forest machine learning module.

19. The computer-implemented training method for a machine learning module for implementing a method according to claim 17 comprising:
A step of providing a training data set, wherein each training data associates:
Values representative of environmental parameters associated with collecting a water sample;
A value representative of a quantity of the viable bacteria, comprising viable culturable and non-culturable bacteria; and
A value representative of a quantity of the viable non-culturable bacteria in the water sample;
A training step for the machine learning module to infer, for each data set, the value representative of a quantity of the viable culturable bacteria from the associated environmental parameters and the value representative of the quantity of the viable bacteria, comprising viable culturable and non-culturable bacteria.

20. An device for estimating a parameter representative of a quantity of the viable culturable bacteria indicative of fecal contamination in a collected water sample, comprising:
Means for acquiring a plurality of values representative of environmental parameters associated with collecting the water sample;
Means for acquiring a quantification of the viable bacteria present in the collected water sample;
An implementation unit for a statistical regression module suitable for estimating a value representative of the quantity of the viable culturable bacteria according to the values representative of the acquired environmental parameters and the quantification of viable bacteria in the water sample.

21. A training device for a machine learning module for implementing a method according to claim 17, comprising:
Means for providing a training data set, wherein each training data associates:
Values representative of environmental parameters associated with collecting a water sample;
A value representative of a quantity of the viable bacteria, comprising viable culturable and non-culturable bacteria; and
A value representative of a quantity of the viable non-culturable bacteria in the water sample;
A training unit for the machine learning module to infer, for each data set, the value representative of a quantity of the viable culturable bacteria from the associated environmental parameters and the value representative of a quantity of the viable bacteria, comprising viable culturable and non-culturable bacteria.
